(19) United States
(12) Patent Application Publication
(10) Pub. No.: US 2015/0196396 A1
(43) Pub. Date: Jul. 16, 2015

(54) DEVICE AND METHOD OF USE FOR A HORIZONTAL THORACIC EXPANDER

(71) Applicant: Robert Powell Thomas, San Antonio, TX (US)

(72) Inventor: Robert Powell Thomas, San Antonio, TX (US)

(21) Appl. No.: 14/598,207

(22) Filed: Jan. 15, 2015

Related U.S. Application Data
(60) Provisional application No. 61/927,526, filed on Jan. 15, 2014.

(51) Int. Cl. A61F 2/28; A61B 17/80

(52) U.S. Cl. CPC A61F 2/28; A61B 17/8076; A61F 2002/3055

(57) ABSTRACT

Disclosed herein is a device and method of use for a thoracic expander. More specifically, the present invention generally relates to a device and method of use for a horizontal thoracic expander, which allows for the creation of thoracic volume in a patient by expanding the thoracic cavity in the horizontal plane. In one embodiment the device is comprised of a housing element and at least one extending element. In another embodiment, the device is comprised of two opposing extending elements. In yet another embodiment, the opposing extending elements are also comprised of interchangeable attachment elements that are positioned on the distal ends of the extending elements. In yet another embodiment, the device is also comprised of a gear element, which is coupled to the extending elements and a control element allowing for the displacement of the extending arms in opposing horizontal directions.

### CROSS-REFERENCE TO RELATED APPLICATIONS

This application claims the benefit under Title 35 United States Code §119(e) of U.S. Provisional Patent Application Ser. No.: 61/927,526; Filed: Jan. 15, 2014, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use for a thoracic expander. More specifically, the present invention generally relates to a device and method of use for a horizontal thoracic expander, which allows for the creation of thoracic volume in a patient by expanding the thoracic cavity in the horizontal plane.

### BACKGROUND OF THE INVENTION

Without limiting the scope of the disclosed device, the background is described in connection with a novel device and method of use for a horizontal thoracic expander, which allows for the creation of thoracic volume in a patient by expanding the thoracic cavity in the horizontal plane.

Respiratory insufficiency may be caused by numerous conditions such as Jeune's syndrome, Jarcho-Levin syndrome, and other abnormalities which result in reduced dimensions of the thorax thereby causing restrictions of the lungs by preventing normal intercostal muscle excursion. Jeune's syndrome, or asphyxiating thoracic dystrophy is a congenital deformity of the chest wall, characterized by an extremely small chest cage that creates insufficient space to allow for proper and efficient breathing. Other deformities associated with this disease are abnormalities of the pelvis and phalanges. Jarcho-Levin syndrome is a genetic disorder characterized by distinctive malformations of bones of the spinal column and the ribs. Other overlapping diseases are Barnes syndrome, Ellis-van Creveld syndrome, Schwachman-Diamond syndrome, Sensenbrenner syndrome, and other short rib polydactyly syndromes. With these and other similar conditions, the individual may develop a symptomatology range from intermittent bouts of respiratory distress to severe respiratory insufficiency requiring breathing assistance.

To help alleviate these issues, several approaches in the prior art have been taken to assist patients. These approaches all involve expanding the thoracic cavity to alleviate respiratory distress. The following examples are some of the common ones utilized today.

As a first example, vertical prosthetic titanium ribs are utilized for vertical rib expansion. This approach has been shown to be more useful for respiratory insufficiency with scoliosis and has a history of life threatening complications with no documentation of increased lung capacity.

As a second example, the Nuss procedure or technique has also been performed. For this procedure, two bent bars are inserted into the thoracic cavity from each lateral intercostal space in the midaxillary line, and pulled out from the intercostal cartilage space around the lower end of the sternum. By rotating the bar one hundred eighty degrees, the concaved chest wall is tilted upwards. The drawback to this approach is the invasiveness, likelihood of puncturing other organs, and the long recovery time.

As a third example, the lateral thoracic expansion approach has also been used. In this approach, ribs four through nine are divided in a staggered fashion with the long ends of the fifth and sixth as well as the seventh and eighths ribs being opposed and secured with titanium plates. While this approach allows healing of bone-to-bone reinforced by regeneration of bone in newly exposed periosteum and thus ultimately not dependent on prosthetic material to maintain expansion, the new rib configuration is unstable from the torque caused by the realignment of the adjacent ribs. These torque forces have caused the titanium support plates to fracture and lead to further complications.

While all of the aforementioned devices and approaches may fulfill their unique purposes, none of them fulfill the need for a practical, effective, and efficient means for creating thoracic volume in a patient by expanding the thoracic cavity in the horizontal plane.

Therefore, the present invention proposes a novel device and method of use for a horizontal thoracic expander, which allows for the creation of thoracic volume in a patient by expanding the thoracic cavity in the horizontal plane and allowing for adjustments without removal and insertion of additional prostheses.

### BRIEF SUMMARY OF THE INVENTION

The present invention, therefore, provides for a device and method of use for expanding the thoracic cavity. More specifically, the present invention generally relates to a device and method of use for a horizontal thoracic expander, which allows for the creation of thoracic volume in a patient by expanding the thoracic cavity in the horizontal plane. In one embodiment the device is comprised of a housing element and at least one extending element. In another embodiment, the device is comprised of two opposing extending elements. In yet another embodiment, the opposing elements are also comprised of interchangeable attachment elements that are positioned on the distal ends of the extending elements. In yet another embodiment, the device is also comprised of a gear element, which is coupled to the extending elements and a control element allowing for the displacement of the extending arms in opposing horizontal directions.

In summary, the present invention discloses a novel device and method of use for a horizontal thoracic expander, which allows the creation of thoracic volume in a patient by expanding the thoracic cavity in the horizontal plane and in an embodiment allowing for adjustments without removal and insertion of additional prostheses.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a top side exploded perspective view of the horizontal thoracic expander device in accordance with embodiments of the disclosure;

FIG. 2 is a top side perspective view of the horizontal thoracic expander device in accordance with embodiments of the disclosure;

FIG. 3 is a front side view of the horizontal thoracic expander device in accordance with embodiments of the disclosure;

FIG. 4 is a left side view of the horizontal thoracic expander device in accordance with embodiments of the disclosure;

FIG. 5 is a top side of the horizontal thoracic expander device in accordance with embodiments of the disclosure;

FIG. 6 is a top side of the horizontal thoracic expander device illustrating the expansion mechanism in accordance with embodiments of the disclosure;

FIG. 7 is a front side view of the thoracic cage in accordance with embodiments of the disclosure;

FIG. 8A is a front side view of the right side of the thoracic cage illustrating the removal of portions of the rib and costal cartilage in accordance with embodiments of the disclosure;

FIG. 8B is a front side environmental view of the right side of the thoracic cage illustrating the attachment of the horizontal thoracic expander where portions of the rib and costal cartilage have been removed in accordance with embodiments of the disclosure;

FIG. 8C is a front side environmental view of the right side of the thoracic cage illustrating the expansion of the horizontal thoracic expander in accordance with embodiments of the disclosure; and

FIG. 9 is a top side exploded perspective view of the horizontal thoracic expander device illustrating an alternative attachment in accordance with embodiments of the disclosure.

### DETAILED DESCRIPTION OF THE INVENTION

Disclosed herein is an improved device and method of use directed to expanding the thoracic cavity. The numerous innovative teachings of the present invention will be described with particular reference to several embodiments (by way of example, and not of limitation).

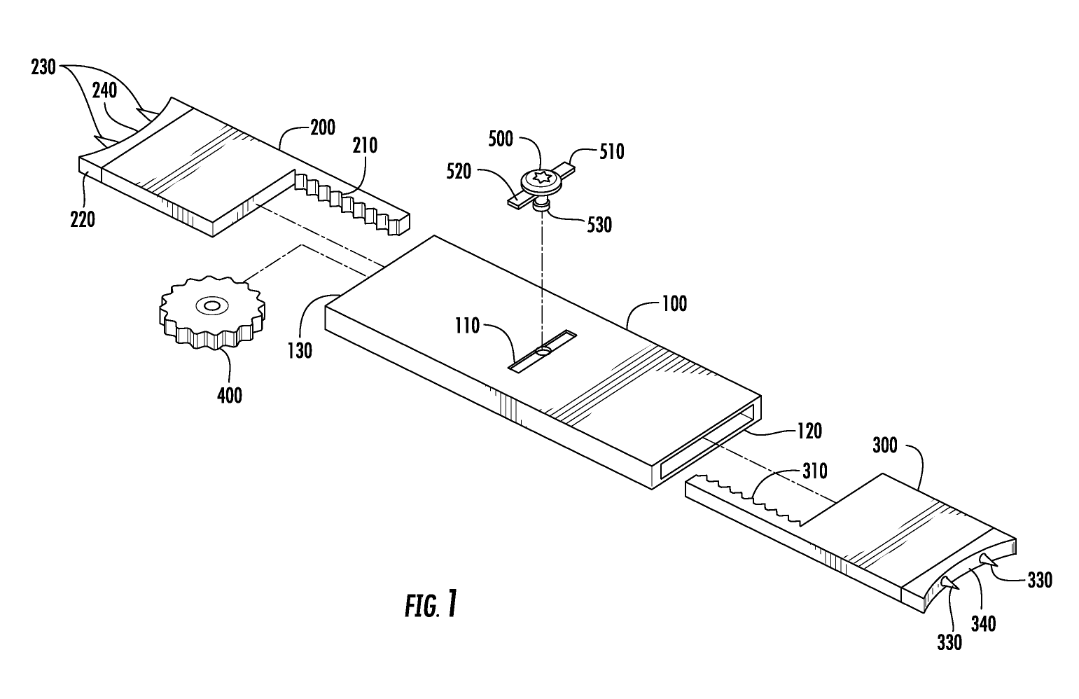

Reference is first made to FIG. 1, a top side exploded perspective view of the horizontal thoracic expander device in accordance with embodiments of the disclosure. In this illustration the components that make up the device are shown. In one embodiment the device is comprised of a housing element 100 and at least one extending element 200, 300 slidably connected to the housing element. In an embodiment, the housing element 100 allows for the sliding of the extending element 200, 300 through the left side opening 130 or the right side opening 230 of the housing element 100. These openings 130, 230 extend completely though the housing element 100 forming an internal hollow portion within the housing element 100 from one end to the other. In an embodiment, the extending element 200, 300 has a gear portion 210, 310 that allows the extending element 200, 300 to be advanced in the left and right direction by having the gear portions 210, 310 engaged with the teeth of the gear element 400. In another embodiment, the device is comprised of a left side extending element 200 and a right side extending element 300, which are opposed to each other in their movement direction. While gears are used and discussed in an embodiment, alternative embodiments may be employed to achieve the movement of the extending elements 200, 300. For example, springs may be housed in the housing element 100 to supply a movement force to the extending elements 200, 300. In yet another embodiment, the opposing extending elements 200, 300 are also comprised of interchangeable attachment elements 220, 320 that are positioned on the distal ends of the extending elements 200, 300. The interchangeable attachment elements 220, 320 assist the extending elements 200, 300 in attaching to the ribs and/or costal cartilage. The interchangeable attachment elements 220, 320 may take on various shapes in order to assist in attaching and/or securing a fit to the ribs and/or costal cartilage. In one embodiment, the interchangeable attachment elements 220, 320 are additionally comprised of teeth or spike structures 230, 330 on the face 240, 340 of the interchangeable attachment elements 220, 320. These teeth 230, 330 structures assist in attaching by penetrating into the middle or marrow of the bone. The interchangeable attachment elements 200, 300 in an embodiment may be interchanged depending on the type of attachment desired. For example, the face portion 240, 340 may be concaved, flat, convex, and may also contain other structures to aid in attachment such as the teeth 230, 330 aforementioned. In yet another embodiment, the device is also comprised of a gear element 400, which is coupled to the extending elements 200, 300 at the gear portions 210, 310 and a control element 500 allowing the displacement of the extending elements 200, 300 in opposing horizontal directions. In an embodiment, the control element 500 has a star screw head and two extending arms 510, 520 with a gear attachment portion 530. In an embodiment, the housing element 100 is also comprised of a control element lock 110. In an embodiment, the control element lock 110 is a control element locking cavity 110. This cavity 110 allows the extending arms 510, 520 to drop into the housing element 100 and lock the control element 500 in position thereby disallowing any rotation of the control element 500 to extend or retract the extending elements 200, 300. The function of the control element locking cavity 110 is to disallow the extending and retracting of the extending elements 200, 300. Alternate embodiments of the function of the control element locking cavity 110 may take numerous forms and are not limited to that one embodiment. For example, a pin may be slid through the control element 500 locking it into place.

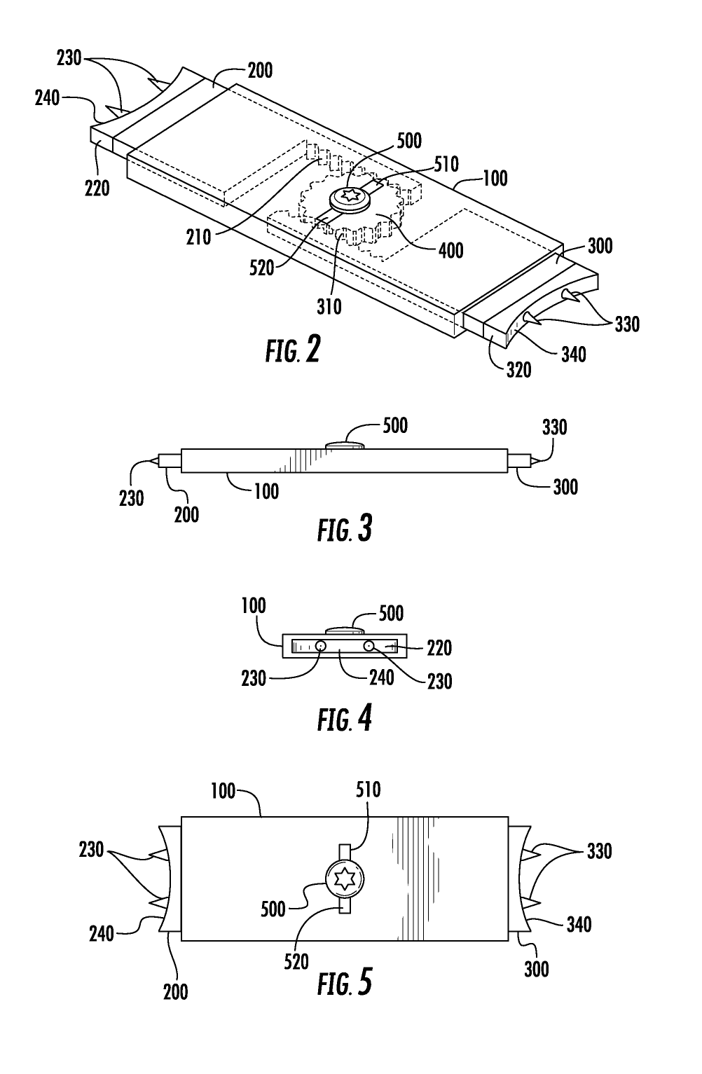

Reference is next made to FIG. 2, a top side perspective view of the horizontal thoracic expander device in accordance with embodiments of the disclosure. Illustrated in this figure are the elements connected and further illustrating the gear element 400 coupled to the extending elements 200, 300 and the gear element 400 also coupled to the control element 500 allowing the displacement or movement of the extending elements 200, 300 in opposing horizontal directions.

Reference is now made to FIG. 3, a front side view of the horizontal thoracic expander device in accordance with embodiments of the disclosure. Illustrated in this figure are the extending elements 200, 300. The left side extending element 200 extends to the left and retracts to the right. The right side extending element 300 extends to the right and retracts to the left.

Reference is next made to FIG. 4, a left side view of the horizontal thoracic expander device in accordance with embodiments of the disclosure. Illustrated in this figure is another view looking at the face portion 240 of the interchangeable attachment elements 220 and the teeth 230 positioned on that face portion.

Reference is now made to FIG. 5, a top side of the horizontal thoracic expander device in accordance with embodiments of the disclosure. Illustrated in this figure is the device in the retracted position. That is, the extending elements 200, 300 are in their innermost position

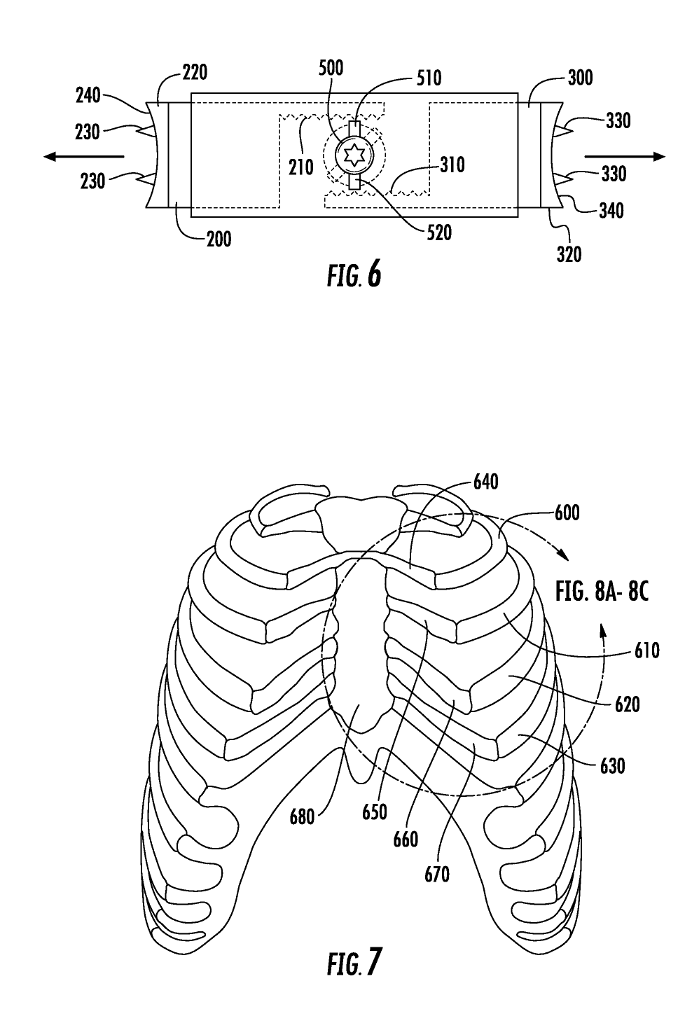

Reference is next made to FIG. 6, a top side of the horizontal thoracic expander device illustrating the expansion mechanism in accordance with embodiments of the disclosure. Illustrated in this figure is the device in the expanded configuration. That is, the left side extending element 200 is being extended to the left and the right side extending element 300 is being extended to the right. This is accomplished by rotating in the counter clockwise direction the control element 500. By rotating the control element 500 (which is coupled to the gear element 400 at its gear attachment portion) in the counter clockwise direction, the extending elements 200, 300 move in this extending direction. To retract the device, the control element is rotated in the clockwise direction.

Reference is now made to FIG. 7, a front side view of the thoracic cage in accordance with embodiments of the disclosure. Illustrated in this figure are the first seven pairs of ribs also referred to as true ribs. Also illustrated are the rib pairs eight and nine also referred to as false ribs. Identified specifically for further discussion are ribs two 600, three 610, four 620, and five 630 as well as costal cartilages for ribs two 640, three 650, four 660, and five 670. In addition, the sternum body 680 is also identified specifically.

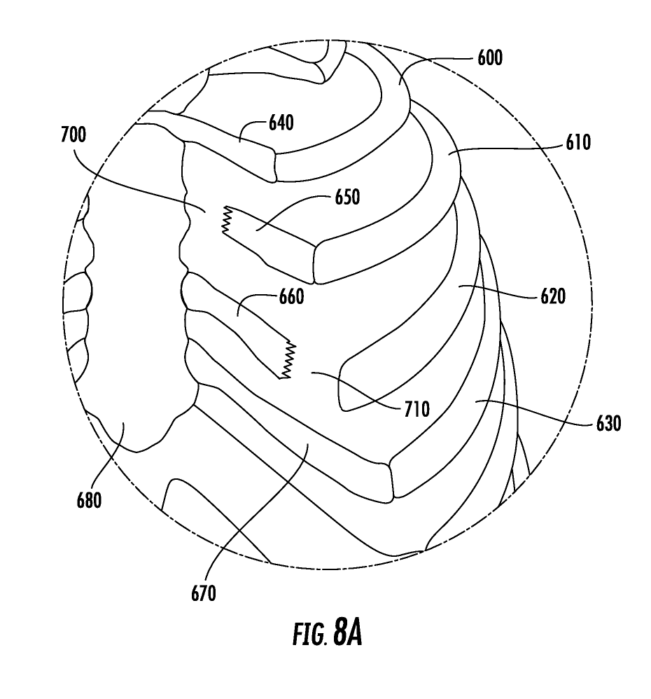

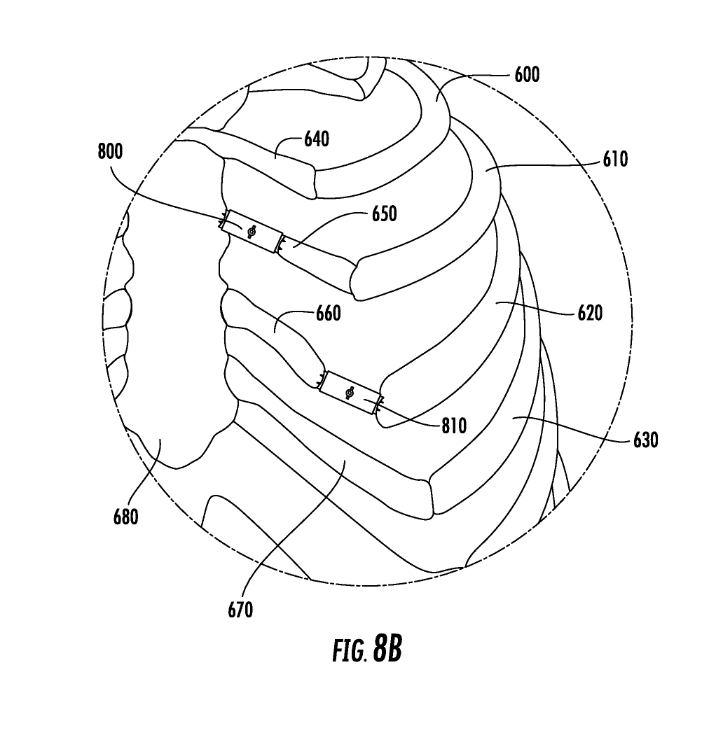

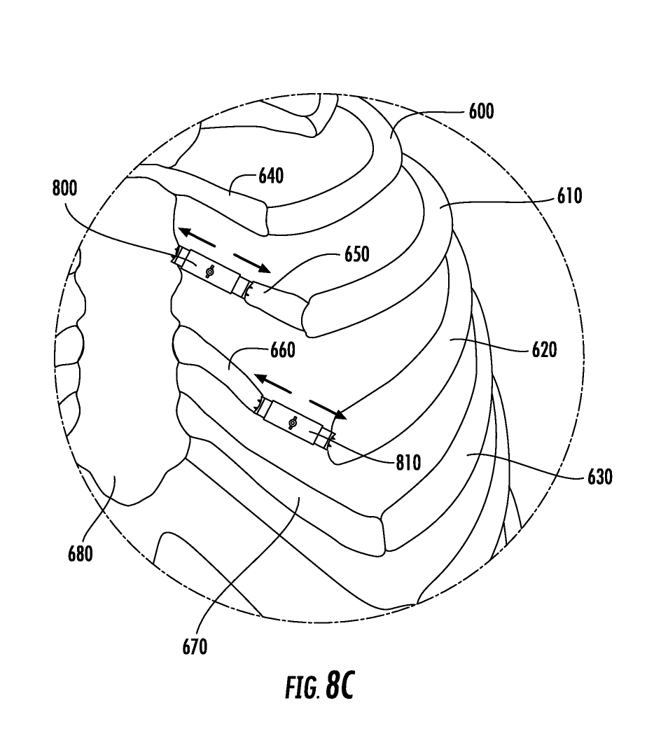

Reference is now made to FIG. 8A, a front side view of the right side of the thoracic cage illustrating the removal of portions of the rib and costal cartilage in accordance with embodiments of the disclosure. Illustrated in this figure is the costal cartilage removed 700, 710 for insertion of the horizontal thoracic expander device. The approach to accomplish this removal task is the same as the Ravitch procedure for pectus excavatum. In the Ravitch procedure, the goal is to remove abnormal rib cartilage while preserving the perichondrium, allowing for the regrowth of rib cartilage to the sternum in a more anatomical fashion. In the method of use of the horizontal thoracic expander, a portion of the costal cartilage connected to the sternum body may be removed 700, a portion of the costal cartilage connected to the rib may be removed 710, or the costal cartilage may be removed in its entirety. As another approach, an anterior rib portion may be removed.

Reference is now made to FIG. 8B, a front side environmental view of the right side of the thoracic cage illustrating the attachment of the horizontal thoracic expander where portions of the rib and costal cartilage have been removed in accordance with embodiments of the disclosure. Illustrated in this figure is a horizontal thoracic expander 800 having its left extending element 200 attached to the sternum body 680 and its right extending element 300 attached to the costal cartilage 650. Also illustrated in this figure is a horizontal thoracic expander 810 having its left extending element 200 attached to the costal cartilage 660 and its right extending element 300 attached to the rib 620. The interchangeable attachment elements 220, 320 provide the means of attaching as previously discussed. In alternative embodiments the attachment means may also embody stitching, an adhesive, pins, screws, or a sleeve cover.

Reference is now made to FIG. 8C, a front side environmental view of the right side of the thoracic cage illustrating the expansion of the horizontal thoracic expander in accordance with embodiments of the disclosure. In this illustration the thoracic cavity is shown being expanded in the horizontal direction by having the extending elements 200, 300 extended for the horizontal thoracic expanders 800, 810. That is, the extending elements 200, 300 provide forces in opposing directions as illustrated by the arrows in the figure to expand the thoracic cavity in the horizontal direction.

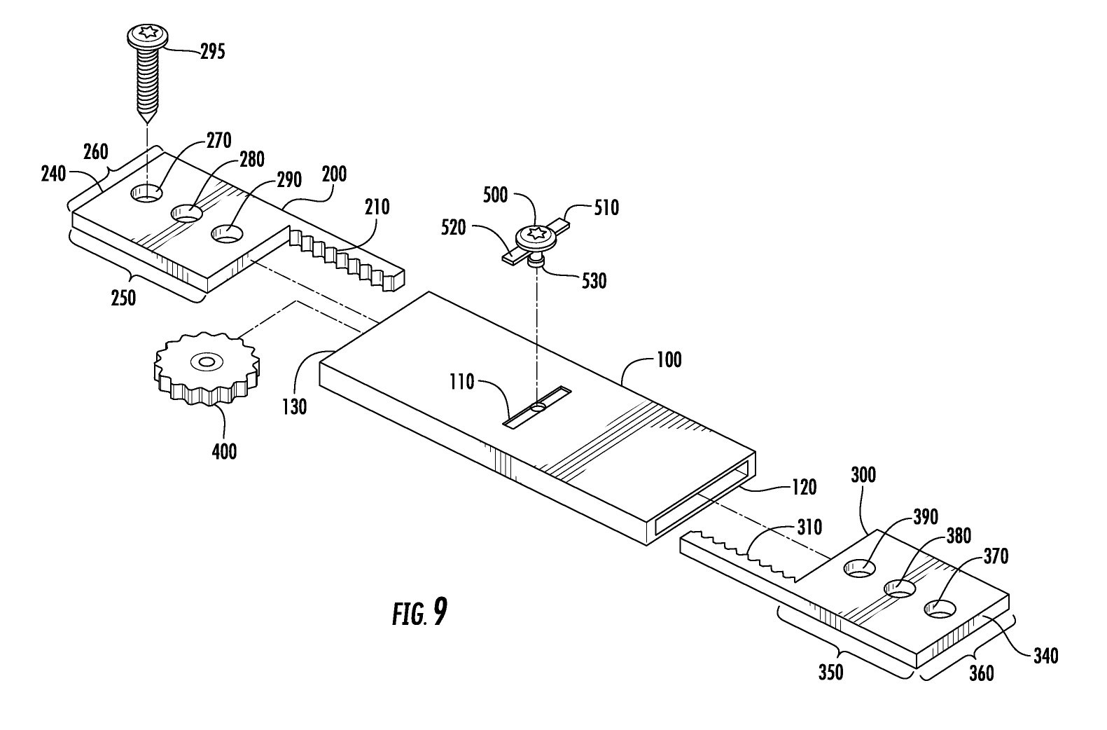

Reference is lastly made to FIG. 9, a top side exploded perspective view of the horizontal thoracic expander device illustrating an alternative attachment by the extending elements 200, 300 in accordance with embodiments of the disclosure. Illustrated are extending elements 200, 300 without attachment elements 220, 320. The left side extending element 200 has a width 260 and a length 250 forming a bar portion which has at least one attachment hole 270, 280, 290. The right side extending element 300 has a width 360 and a length 350 forming a bar portion which has at least one attachment hole 370, 380, 390. The lengths 250, 350 and widths 260, 360 may vary (changing the bar portion size) based on the patient size and application. Instead of using attachment elements 220, 320 to couple the horizontal thoracic expander device to the patient, the bar portion is of sufficient length to overlay the attachment area of the patient and a pin, screw, or similar structure 295 is used to attach the horizontal thoracic expander device to the patient utilizing an attachment hole 270, 280, 290, 370, 380, 390. That is, in an embodiment, the screw 295 is threaded through an attachment hole 270, 280, 290, 370, 380, 390 into the patient's bone or cartilage.

In brief, the invention is directed to a device and method of use for expanding the thoracic cavity.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method of use, except as may be outlined in the claims.

Alternative applications for this invention include using this device or method of use in any application where the thoracic cavity needs to be expanded or an area where a bone and/or cartilage structure needs to be expanded. Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods of use disclosed and claimed herein can be made and executed without undue experimentation in light of the present disclosure. While the device and methods of use of this invention have been described in terms of preferred embodiments, it will be apparent to those skilled in the art that variations may be applied to the device and/or methods of use and in the steps or in the sequence of steps of the method of use described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A horizontal thoracic expander device comprising:
a housing element; and
at least one extending element slidably connected to said housing element.

2. The device of claim 1, wherein said housing element is further comprised of a hollow portion.

3. The device of claim 2, wherein said extending elements are positioned within said housing element's hollow portion.

4. The device of claim 3, wherein said device has two said extending elements that extend and retract in opposite directions.

5. The device of claim 3, wherein said extending elements retract and extend through the left and right side housing element openings.

6. The device of claim 5, wherein said extending elements are further comprised of attachment elements.

7. The device of claim 6, wherein said attachment elements are positioned at the distal ends of said extending elements.

8. The device of claim 7, wherein said attachment elements are interchangeable.

9. The device of claim 6, wherein said attachment elements are in the group consisting of spikes, teeth, stitching, adhesive, pins, screws, or a sleeve cover.

10. The device of claim 6, wherein said attachment element's face is concave.

11. The device of claim 6, wherein said attachment element's face is flat.

12. The device of claim 6, wherein said attachment element's face is convex.

13. The device of claim 4, wherein said housing element is further comprised of a gear element coupled to said extending elements.

14. The device of claim 13, wherein said housing element is further comprised of a control element coupled to said gear element.

15. The device of claim 14, wherein said housing element is further comprised of a control element lock to restrict the movement of said extending elements.

16. The device of claim 15, wherein said control element lock is a locking cavity.

17. The device of claim 14, wherein said control element is a star screw being further comprised of a gear attachment portion.

18. The device of claim 17, wherein said housing element is further comprised of a control element lock to restrict the movement of said extending elements.

19. The device of claim 18, wherein said locking element is further comprised of extending arms and wherein said control element lock is a locking cavity wherein said extending arms may be secured within said locking cavity.

20. A horizontal thoracic expander device comprising:
a housing element; and
at least one extending element slidably connected to said housing element;
and wherein said housing element is further comprised of a hollow portion;
and wherein said extending elements extend and retract in opposite directions through the left and right side housing element openings;
and wherein said extending elements are further comprised of attachment holes for attaching said device.
